(19) United States
(12) Patent Application Publication
(10) Pub. No.: US 2003/0022799 A1
(43) Pub. Date: Jan. 30, 2003

(54) A SHAMPOO FOAMING COMPOSITION WHICH COMPRISES AN ALKYL ETHER SULFATE, A SORBITAN DERIVATIVE, A BETAINE, AN ALKYLAMIDO ALKYLAMINE, AN ALKOXYLATED CARBOXYLIC ACID, AND AN ORGANIC SALT

(76) Inventors: Robert M. Alvarado, Glenview, IL (US); Richard A. Abbott, Downers Grove, IL (US); Chaitanya Umedbhai Patel, Glen Ellyn, IL (US)

Correspondence Address: UNILEVER, PATENT DEPARTMENT, 45 RIVER ROAD, EDGEWATER, NJ, 07020, US

(21) Appl. No.: 09/916,133

(22) Filed: Jul. 27, 2001

(51) Int. Cl. A61K 7/075

(52) U.S. Cl. 510/119; 510/121; 510/125; 510/466

(57) ABSTRACT

A foamable, shampoo composition for cleansing hair which comprises:

(i) about 0.005 to about 5% of a cationic deposition polymer having a cationic charge density of about 0.1 to about 4.0 meq/gram;

(ii) about 10 to about 20% of an anionic surfactant which is selected from the group consisting of an alkyl ether sulfate with at least about two moles of ethoxylation, and a neutralized alkyl ether sulfate with at least about two moles of ethoxylation; and mixtures thereof;

(iii) about 0.5 to about 1.0% of an organic salt of a carboxylic acid;

(iv) about 6 to 15% sorbitan derivative;

(v) about 3 to about 6% of a zwitterionic, surface active compound which includes quaternized alkyl or substituted alkyl derivatives of N,N-dimethylycine;

(vi) about 0.25 to about 5% of an amphoteric surfactant;

(vii) about 0.75 to about 1.5% of an alkoxylated carboxylic acid

(viii) about 0.1 to about 5% of a silicone copolyol

(ix) optionally an aerosol propellant; and

(x) water.

### BACKGROUND OF THE INVENTION

Hair care products that are shampoos, can suffer from the following two drawbacks: firstly, the cleansing surfactant can be irritating to the eyes (in the event, for example, that shampoo composition is washed into the eyes during rinsing). Secondly, the shampoo composition can be irritating to the skin. In addition, when young children or infants use a shampoo composition, the traditional shampoo liquid compositions are not always an easy form for such children and infants to handle.

It is an object of the present invention to provide hair care shampoo compositions which are not irritating or which cause very little irritation to the skin and to the eyes. It is also an object of the present invention to provide a shampoo composition in the form of an aerosol or nonaerosol mousse.

Publications which relate to the area of technology of the invention are as follows:

U.S. Pat. No. 6,096,702 discloses clear solutions or gels containing a volatile hydrocarbon, surfactants and a polyalkylene oxide block copolymer coupling agent that do not separate, but rather due to solubilization of the volatile hydrocarbon by the coupling agent, provide acceptable consumer products.

U.S. Pat. No. 6,110,451 discloses a keratin conditioning composition that contains (a) a surfactant component that can contain anionic surfactants, amphoteric surfactants, cationic surfactants, nonionic surfactants and/or zwitterionic surfactants, (b) a water soluble, organic, ampholytic polymer conditioning agent, (c) a water soluble, organic, cationic polymer conditioning agent; (d) optionally a water insoluble liquid; and (e) an aqueous carrier. The conditioning composition optionally contains a silicone and/or an organic, water insoluble, liquid. The conditioning composition according to the present invention is useful in cleaning and/or conditioning keratin based substrates, such as hair, skin, and nails.

WO 9932070 discloses a mousse-forming cleansing shampoo composition having improved conditioning performance comprising:

(A) a foamable concentrate comprising:

(i) at least one surfactant;

(ii) emulsified particles of a conditioning agent having a particle size of less than or equal to 1 micron;

(iii) a deposition polymer for the emulsified particles;

(iv) an aqueous carrier; and

(B) an aerosol propellant.

### SUMMARY OF THE INVENTION

The invention relates to a foamable, shampoo composition for cleansing hair which comprises:

(i) about 0.005 to about 5% of a cationic deposition polymer having a cationic charge density of about 0.1 to about 4.0 meq/gram;

(ii) about 10 to about 20% of an anionic surfactant which is selected from the group consisting of an alkyl ether sulfate with at least about two moles of ethoxylation, and a neutralized alkyl ether sulfate with at least about two moles of ethoxylation; and mixtures thereof;

(iii) about 0.5 to about 1.0% of an organic salt of a carboxylic acid;

(iv) about 6 to 15% sorbitan derivative;

(v) about 3 to about 6% of a zwitterionic, surface active compound which includes quaternized alkyl or substituted alkyl derivatives of N,N-dimethylycine;

(vi) about 0.25 to about 5% of an amphoteric surfactant;

(vii) about 0.75 to about 1.5% of an alkoxylated carboxylic acid

(viii) about 0.1 to about 5% of a silicone copolyol

(ix) optionally an aerosol propellant; and

(x) water.

The invention is also directed to a method for shampooing hair which comprises contacting said hair with a composition of the invention.

### DETAILED DESCRIPTION OF THE INVENTION

As used herein, % means weight % of the total composition unless otherwise designated. Ingredients are given in weight % as against the total composition, unless otherwise specified “Foamable” compositions are compositions which foam upon being dispensed with an aerosol or nonaerosol container. Not irritating to the eyes and to the skin means that the composition causes little or no discomfort to the human eye or to the skin. Eye irritation can be measured by the following tests: Flourescein Leakage Assay, Epiocular Model, direct eye sting tests in the human eye, Soap Chamber Test, Repeated Insult Patch Test, and exaggerated consumer use testing. The compositions of the invention are made using known ingredients or with ingredients analogous to those known in the art. The packages or containers to be used with the compositions of the invention are made using known processes and materials or by processes and materials which are analogous to those known in the art.

As noted above, hair care products that are shampoos, can suffer from the following two drawbacks: firstly, the cleansing surfactant can be irritating to the eyes (in the event, for example, that shampoo composition is washed into the eyes during rinsing). Secondly, the shampoo composition can be irritating to the skin. In addition, when young children or infants use a shampoo composition, the traditional shampoo liquid compositions are not always an easy form for such children and infants to handle.

It is an object of the present invention to provide hair care shampoo compositions which are not irritating or which cause very little irritation to the skin and to the eyes. It is also an object of the present invention to provide a shampoo composition in the form of an aerosol or nonaerosol mousses.

To provide a shampoo composition that is very mild to the skin and to the eyes, blends of anionic, nonionic and amphoteric surfactants are used. One desirable feature of the compositions of the present invention are the surfactant systems which are employed. These surfactant systems comprise an alkyl ether sulfate, a sorbitan derivative, a betaine, an alkylamido alkylamine, an alkoxylated carboxylic acid, and an organic salt these materials are described in more detail in the description of the ingredients which can be used in the compositions of the invention and which is set forth below.

Another desirable feature of the compositions of the invention, is that they are foamable. That is, compositions of the invention are dispensed, for example, as a foam from an aerosol or nonaerosol can. Such a foamable composition provides a product form that is easier, for example, for children to use than are traditional shampoo liquids. Children can control the amount of foam dispensed more easily than they can control liquids that are poured from a bottle. Liquids which are poured from a bottle can run through a child's hands while, by contrast, a set amount of foam can easily be placed into a child's hair or hands and can be easily manipulated by the child's hands. In addition, foams tend to be more fun for children to use, since they are often light and even fluffy in appearance. Finally, foams are less likely to accidentally run into a child's eyes upon rinsing the shampoo composition from the hair and consequently they are less likely to even contact a child' eyes and thereby cause discomfort or irritation. (But it should be noted that the compositions of the present invention are so mild, that even if they are rinsed into the eyes, they will cause little or no eye irritation.)

Of course the mild, foamable compositions of the invention are also suitable for use by adults.

The invention relates to a foamable, shampoo composition for cleansing hair which comprises:

(i) about 0.005 to about 5% of a cationic deposition polymer having a cationic charge density of about 0.1 to about 4.0 meq/gram;

(ii) about 10 to about 20% of an anionic surfactant which is selected from the group consisting of an alkyl ether sulfate with at least about two moles of ethoxylation, and a neutralized alkyl ether sulfate with at least about two moles of ethoxylation; and mixtures thereof;

(iii) about 0.5 to about 1.0% of an organic salt of a carboxylic acid;

(iv) about 6 to 15% sorbitan derivative;

(v) about 3 to about 6% of a zwifterionic, surface active compound which includes quaternized alkyl or substituted alkyl derivatives of N,N-dimethylycine;

(vi) about 2.5 to about 5% of an amphoteric surfactant;

(vii) about 0.75 to about 1.5% of an alkoxylated carboxylic acid

(viii) about 0.1 to about 5% of a silicone copolyol

(ix) optionally an aerosol propellant; and

(x) water.

More specifically, the invention relates to a foamable, shampoo composition for cleansing hair which comprises:

(i) about 0.1 to about 1% of a cationic deposition polymer having a cationic charge density of about 0.1 to about 4.0 meq/gram;

(ii) about 12 to about 18% of an anionic surfactant which is selected from the group consisting of an alkyl ether sulfate with at least about two moles of ethoxylation, and a neutralized alkyl ether sulfate with at least about two moles of ethoxylation; and mixtures thereof;

(iii) about 0.6 to about 0.9% of an organic salt of a carboxylic acid;

(iv) about 6 to about 15% of sorbitan derivative;

(v) about 4 to about 5% of a zwitterionic, surface active compound which includes quaternized alkyl or substituted alkyl derivatives of N,N-dimethylycine;

(vi) about 3 to about 5% of an amphoteric surfactant;

(vii) about 0.75 to about 1.5% of an alkoxylated carboxylic acid

(viii) about 0.25 to about 1% of a silicone copolyol

(ix) optionally an aerosol propellant; and

(x) water.

What follows now is a detailed description of the ingredients which can be used in forming the compositions of the invention.

Cationic Deposition Polymer

A water soluble, cationic deposition polymer may be used in the compositions of the present invention. It will generally be present at levels from 0.01 to 5%. The polymer may be a homopolymer or be formed from two or more types of monomers. The molecular weight of the polymer will generally be between about 5000 and about 10,000,000, typically at least about 10,000 and preferably in the range of about 100,000 to about 2,000,000. The cationic polymers will have cationic nitrogen containing groups such as quaternary ammonium or protonated amino groups, or a mixture thereof.

The cationic charge density may be at least about 0.1 meg/g, preferably above 0.8 or higher. The cationic charge density should not exceed about 4 meg/g. It is preferably less than 3 and more preferably less than 2 meg/g. The charge density can be measured using the Kjeldahl method and should be within the above limits at desired pH of use, which will in general be from about 3 to 9 and preferably between 4 and 8.

The cationic nitrogen-containing group will generally be present as a substituent on a fraction of the total monomer units of the cationic polymer. Thus when the cationic polymer is not a homopolymer it can contain spacer non-cationic monomer units. Such polymers are described in the CTFA Cosmetic Ingredient Directory, 3rd edition.

Suitable cationic polymers include, for example, copolymers of vinyl monomers having cationic amine or quaternary ammonium functionalities with water soluble spacer monomers such as (meth)acrylamide, alkyl and dialkyl (meth)acrylamides, alkyl (meth)acrylate, vinyl caprolactone and vinyl pyrrolidine. The alkyl and dialkyl substituted monomers preferably have C1-C7 alkyl groups, more preferably C1-3 alkyl groups. Other suitable spacers include vinyl esters, vinyl alcohol, maleic anhydride, propylene glycol and ethylene glycol.

The cationic amines can be primary, secondary or tertiary amines, depending upon the particular species and the pH of the shampoo. In general secondary and tertiary amines, especially tertiary, are preferred.

Amine substituted vinyl monomers and amines can be polymerized in the amine form and then converted to ammonium by quaternization.

Suitable cationic amino and quaternary ammonium monomers include, for example, vinyl compounds substituted with dialkyl aminoalkyl acrylate, dialkylamino alkylmethacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidine, e.g., alkyl vinyl imidazolium, and quaternized pyrrolidine, e.g., alkyl vinyl imidazolium, alkyl vinyl pyridinium, alkyl vinyl pyrrolidine salts. The alkyl portions of these, monomers are preferably lower alkyls such as the C1-C3, alkyls, more preferably C1 and C2 alkyls.

Suitable amine-substituted vinyl monomers for use herein include dialkylaminoalkyl acrylate, dialkylaminoalkyl methacrylate, dialkylaminoalkyl acrylamide, and dialkylaminoalkyl methacrylamide, wherein the alkyl groups are preferably C1-C7 hydrocarbyls, more preferably C1-Calkyls.

The cationic polymers hereof can comprise mixtures of monomer units derived from amine-and/or quaternary ammonium-substituted monomer and/or compatible spacer monomers.

Suitable cationic hair conditioning polymers include, for example: copolymers of 1-vinlyl-2-pyrrolidine and 1-vinyl-3-methyl-imidazolium salt (e.g., Chloride salt) (referred to in the industry by the Cosmetic, Toiletry, and Fragrance Association, “CTFA”. as Polyquaternium-16) such as those commercially available from BASF Wyandotte Corp. (Parsippany, N.J., USA) under the LUVIQUAT tradename (e. g., LUVIQUAT FC 370); copolymers of 1-vinyl-2-pyrrolidine and dimethylaminoethyl methacrylate (referred to in the industry by CTFA as Polyquaternium-11) such as those commercially from Gar Corporation (Wayne, N.J., USA) under the GAFQUAT tradename (e.g., GAFQUAT 755N); cationic diallyl quaternary ammonium-containing polymers. including, for example, dimethyidiallyammonium chloride homopolymer and copolymers of acrylamide and dimethyidiallyammonium chloride, referred to in the industry (CTFA) as Polyquaternium 6 and Polyquaternium 7, respectively; and mineral acid salts of amino-alkyl esters of homo-and co- polymers of unsaturated carboxylic acids having from 3 to 5 carbon atoms, as described in U.S. Pat. No. 4,009,256, incorporated herein by reference.

Other cationic polymers that can be used include polysaccharide polymers, such as cationic cellulose derivatives and cationic starch derivatives.

Anionic Surfactant

An anionic surfactant may be used in compositions of the present invention at about 10 to 20% in our formulation).

Anionic surfactants with at least two moles of ethylene oxide may be used in the compositions of the present invention. There also may be used in compositions of the present invention alkyl ether sulfates with at least two moles of ethoxylation and neutralized with an appropriate base. Nonlimiting examples of the base which may be employed include sodium hydroxide, ammonium hydroxide, magnesium hydroxide, monoethanolamine, diethanolamine, triethanolamine, monoisopropanolamine, or triisopropanolamine, and mixtures thereof. The anionic surfactants which may be used in compositions of the invention may have an aliphatic fatty alkyl chain derived from either natural or synthetic sources and said alkyl chains may have from 6 to 18 carbon atoms in the alkyl chain, wherein the alkyl chain may be either straight-chained or branched.

Examples of anionic surfactants may be found in the CTFA Cosmetic Ingredient Dictionary, 3^{rd} Edition. These include, but are not limited to, the following anionic surfactants with at least two moles of ethoxylation: Ammonium C12-15 Pareth Sulfate, Ammonium Laureth Sulfate, DEA-Laureth Sulfate, DEA-Myreth Sulfate, Magnesium Laureth Sulfate, Magnesium Myreth Sulfate, Magnesium Oleth Sulfate, MEA-Laureth Sulfate, MIPA C12-15 Pareth Sulfate, MIPA-Laureth Sulfate, Sodium Coceth Sulfate, Sodium C12-13 Pareth Sulfate, Sodium C12-15 Pareth Sulfate, Sodium Deceth Sulfate, Sodium Laneth Sulfate, Sodium Laureth Sulfate, Sodium Myreth Sulfate, Sodium Oleth Sulfate, Sodium Trideceth Sulfate, TEA-Laneth Sulfate, TEA-Laureth Sulfate, and TIPA-Laureth Sulfate.

Organic Salt of a Carboxylic Acid

The compositions of the present invention may also contain about 0.5 to 1.0% an organic salt of a carboxylic acid.

An organic salt of carboxylic acid is an organic salt of carboxylic acid with at least two moles of ethylene oxide. The organic salt of carboxylic acid with at least two moles of ethoxylation may further be neutralized with an appropriate base. Nonlimiting examples of such bases include sodium hydroxide, ammonium hydroxide, magnesium hydroxide, or monoethanolamine.

An organic salt of a carboxylic acid may further have an aliphatic fafty alkyl chain derived from either natural or synthetic sources. The aliphatic fatty alkyl chain derived from either natural or synthetic sources may from about 6 to about 18 carbon atoms in the alkyl chain which may be either straight-chained or branched.

Examples of these types of compounds can be found in the CTFA Cosmetic Ingredient Dictionary, 3^{rd} Addition. These include, but are not limited to, the following compounds: Ammonium Laureth-6 Carboxylate, Ammonium Laureth-8 Carboxylate, Magnesium Laureth-11 Carboxylate, MEA-Laureth-6 Carboxylate, MEA-PPG-6-Laureth-7-Carboxylate, Sodium Carpryleth-2-Carboxylate, Sodium Capryleth-9-Carboxylate, Sodium Ceteth-13-Carboxylate, Sodium C11-15-Pareth-7 Carboxylate, Sodium C12-15-Pareth-6 Carboxylate, Sodium C12-15-Pareth-7 Carboxylate, Sodium Deceth-2 Carboxylate, Sodium Hexeth-4 Carboxylate, Sodium lsosteareth-6 Carboxylate, Sodium lsosteareth-11 Carboxylate, Sodium Laureth-4 Carboxylate, Sodium Laureth-5 Carboxylate, Sodium Laureth-6 Carboxylate, Sodium Laureth-11 Carboxylate, Sodium Laureth-13 Carboxylate, Sodium Laureth-14 Carboxylate, Sodium Laureth-17 Carboxylate, Sodium Trideceth-3 Carboxylate, Sodium Trideceth-6 Carboxylate, Sodium Trideceth-7 Carboxylate, and Sodium Trideceth-12 Carboxylate.

Sorbitan Derivative;

The compositions of the present invention may contain a sorbitan derivative which may be present at about 5-10% as PEG-80 Sorbitan Laurate and 1-5% as Polysorbate-20).

The sorbitan derivative is ethoxylated, resulting in a polyalkoxylated derivative which possesses four hydroxyl groups. The sorbitan derivative may further be esterified to provide esters which include polysorbates and PEG-sorbitan derivatives.

Examples of these types of compounds can be found in the CTFA Cosmetic Ingredient Dictionary, 3^{rd} Addition.

These include, but are not limited to, the following compounds: PEG-6 Sorbitan Beeswax, PEG-8 Sorbitan Beeswax, PEG-20 Sorbitan Beeswax, PEG-40 Sorbitan Diisostearate, PEG-5 Sorbitan Isostearate, PEG-20 Sorbitan Isostearate, PEG-40 Sorbitan Lanolate, PEG-75 Sorbitan Lanolate, PEG-10 Sorbitan Laurate, PEG-40 Sorbitan Laurate, PEG-44 Sorbitan Laurate, PEG-75 Sorbitan Laurate, PEG-80 Sorbitan Laurate, PEG-3 Sorbitan Oleate, PEG-6 Sorbitan Oleate, PEG-80 Sorbitan Palmitate, PEG-40 Sorbitan Peroleate, PEG-3 Sorbitan Stearate, PEG-6 Sorbitan Stearate, PEG-40 Sorbitan Stearate, PEG-30 Sorbitan Tetraoleate, PEG-40 Sorbitan Tetraoleate, PEG-60 Sorbitan Tetraoleate, PEG-60 Sorbitan Tetrastearate, Polysorbate 20, Polysorbate 21, Polysorbate 40, Polysorbate 60, Polysorbate 61, Polysorbate 65, Polysorbate 80, Polysorbate 81, and Polysorbate 85.

Zwitterionic, Surface Active Compound

A zwitterionic, surface active compound may be present in compositions of the present invention at about 3.0 to about 6.0%.

The zwifterionic, surface-active compound can include the quaternized alkyl or substituted alkyl derivatives of N,N-dimethyl glycine. These compounds are known as betaines. Furthermore, these compounds may contain an alkyl group or an amide-interrupted alkyl group. Furthermore, the alkyl or amide-interrupted alkyl group may be range from 10 to 22 carbon atoms. Furthermore, the alkyl or amide-interrupted alkyl group may be saturated or unsaturated.

Furthermore, the use of a related amphoteric compound derived from amino propane sulfonic acids may also be used.

Nonlimiting examples of these types of compounds can be found in the CTFA Cosmetic Ingredient Dictionary, 3^{rd} Addition and include the following: Almondamidopropyl Betaine, Apricotamidopropyl Betaine, Avocadoamidopropyl Betaine, Babassuamidopropyl Betaine, Behenamidopropyl Betaine, Behenyl Betaine, Betaine, Carnitine, Cetyl Betaine, Cocamidoethyl Betaine, Cocamidopropyl Betaine, Cocamidopropyl Hydroxysultaine, Coco-Betaine, Coco-Hydroxysultaine, Coco/Oleamidopropyl Betaine, Coco-Sultaine, Decyl Betaine, Dimethicone Propyl PG-Betaine, Erucamidopropyl Hydroxysultaine, Hydrogenated Tallow Betaine, Isostearamidopropyl Betaine, Lauramidopropyl Betaine, Lauryl Betaine, Lauryl Hydroxysultaine, Lauryl Sultaine, Minkamidopropyl Betaine, Myristamidopropyl Betaine, Myristyl Betaine, Oleamidopropyl Betaine, Oleamidopropyl Hydroxysultaine, Oleyl Betaine, Olivamidopropyl Betaine, Palmamidopropyl Betaine, Palmitamidopropyl Betaine, Palm Kernelamidopropyl Betaine, Ricinoleamidopropyl Betaine, Sesamidopropyl Betaine, Stearyl Betaine, Tallowamidopropyl Betaine, Tallowamidopropyl Hydroxysultaine, Tallow Betaine, Tallow Dihydroxyethyl Betaine, Undecylenamidopropyl Betaine, and Wheat Germamidopropyl Betaine.

Amphoteric Surfactant

Compositions of the invention may contain about 2.5 to about 5% of an amphoteric surfactant.

Amphoteric surfactants which may be used in the compositions of the invention are alkylamidoamines. These amphoteric surfactants are carboxylated surfactants derived from fatty imidazolines. These surfactants may be monocarboxylated or dicarboxylated. These surfactants are amphoteric because they behave as anionic surfactants above the isoelectric point, cationic when the pH is acidic with respect to this range, and electonically neutral zwifterions within the isoelectric range. These amphoteric surfactants contain at least two sites which are capable of ionization, generally provided by a tertiary amine group (electropositive) and either a carboxylate or a sulfonate group (both electronegative).

These amphoteric surfactants may be produced from the reaction of one or two moles of sodium chloroacetate with the fatty imidazoline.

All of these materials contain a carboxymethyl group and can be regarded as derivatives of acetic acid.

Amphoteric surfactants which can be used the compositions of the invention can also be propionates. These propionates contain a carboxyethyl group in place of the carboxymethyl group, and are described as derivatives of propionic acid. These amphoteric surfactants may be produced from the reaction of a primary alkyl amine with one or two moles of acrylic acid.

Nonlimiting examples of these types of compounds include the following compounds: Cocoamphodiproponic Acid, Cocbetainamidopropyl Amphopropionate, DEA-Cocoamphodipropionate, Disodium Caproamphodiacetate, Disodium Caproamphodipropionate, Disodium Capryloamphodiacetate, Disodium Capryloamphodipropionate, Disodium Cocoamphocarboxyethylhydroxypropylsulfonate, Disodium Cocoamphodiacetate, Disodium Cocoamphodipropionate, Disodium lsostearoamphodiacetate, Disod ium Isostearoamphod ipropionate, Disod ium Lauroamphodiacetate, Disodium Lauroamphodipropionate, Disodium Oleoamphodipropionate, Disodium Stearoamphodiacetate, Disodium Tallowamphodiacetate,Disodium Wheatgermamphodiacetate, Lauroamphodipropionic Acid, Sodium Caproamphoacetate, Sodium Caproamphohydroxypropylsulfonate, Sodium Caproamphopropionate, Sodium Capryloamphoacetate, Sodium Capryloamphohydroxypropylsulfonate, Sodium Capryloamphopropionate, Sodium Cocoamphoacetate, Sodium Cocoamphohydroxypropylsulfonate,Sodium Cocoamphopropionate, Sodium Cornamphopropionate, Sodium Isostearoamphoacetate, Sodium lsostearoamphopropionate, Sodium Lauroamphoacetate, Sodium Lauroamphohydroxypropylsulfonate, Sodium Lauroampho PG-Acetate Phosphate, Sodium Lauroamphopropionate, Sodium Myristoacetate, Sodium Myristopropionate, Sodium Oleoamphoacetate, Sodium Oleoamphohydroxypropylsulfonate, Sodium Oleoamphopropionate, Sodium Ricinoleoamphoacetate, Sodium Stearoamphoacetate, Sodium Stearoamphohydroxypropylsulfonate, Sodium Stearoamphopropionate, Sodium Tallamphopropionate, Sodium Tallowamphoacetate, Sodium Undecylenoamphoacetate, Sodium Undecylenoamphopropionate, Sodium Wheat Germamphoacetate, Trisodium Lauroampho PG-Acetate Phosphate Chloride.

Alkoxylated Carboxylic Acids

Alkoxylated Carboxylic Acids may also be included in the compositions of the invention at 0.75% to about 1.5%. Furthermore, the alkoxylated Carboxylic Acid may be formed from the reaction of a carboxylic acid with either an alkylene oxide or with a preformed polymeric ether. The alkoxylated carboxylic acid may be a monoester, a diester, or a mixture of the two. The alkyl group on the carboxylic acid radical can range from 6 to 22 carbon atoms. The alkyl group on the carboxylic acid radical can be either saturated or unsaturated. The number of polymerized alkoxyl groups can range from 2 to 200. Examples of these types of compounds can be found in the CTFA Cosmetic Ingredient Dictionary, 3^{rd} Edition. Examples of these types of compounds include, but are not limited to, the following: PEG-8 Behenate, PEG-8 Caprate, PEG-8 Caprylate, PEG-8 Caprylate/Caprate, PEG-8 C12-C15 Ester, PEG-5 Cocoate, DEG-8 Dicocoate, PEG-2 Diisononanoate, PEG-8 Diisostearate, PEG-2 Dilaurate, PEG-150 Dilaurate, PEG-2 Dioctanoate, PEG-4 Dioleate, PEG-150 Dioleate, PEG-3 Dipalmitate, PEG-2 Distearate, PEG-150 Distearate, PEG-175 Distearate, PEG-8 Ditallate, PEG-12 Ditallate, PEG-8 Di/Triricinoleate, PEG-15 Hydroxystearate, PEG-6 Isopalmitate, PEG-4 Isostearate, PEG-12 Isostearate, PEG-15 Jojoba Acid, PEG-1 20 Jojoba Acid, PEG-5 Lanolate, PEG-20 Lanolate, PEG-2 Laurate, PEG-150 Laurate, PEG-2 Laurate SE, PEG-6 Laurate/Tartarate, PEG-8 Linoleate, PEG-8 Linolenate, PEG-200 Montanate, PEG-8 Myristate, PEG-20 Myristate, PEG-4 Octanoate, PEG-13 Octanoate, PEG-2 Oleate, PEG-150 Oleate, PEG-2 Oleate SE, PEG-6 Palmitate, PEG-20 Palmitate, PEG-2 Ricinoleate, PEG-9 Ricinoleate, PEG-8 Sesquioleate, PEG-2 Stearate, PEG-150 Stearate, PEG-45 Stearate Phosphate, PEG-2 Stearate SE, PEG-4 Tallate, PEG-20 Tallate, PEG-20 Tallowate, and Polyglyceryl-2-PEG-4 Stearate.

Silicone Copolyols

Silicone copolyols that may be used in the compositions of the invention include dimethicone, which is a dimethylsiloxane polymer having polyoxyethylene and/or polyoxypropylene sidechains, such as DOW CORNING 3225C and 5225C FORMULATION AID, available from Dow Corning Co., Midland, Mich., SILICONE SF-1528, available from General Electric, Waterford, N.Y., ABIL EM 97, available from Goldschmidt Chemical Corporation, Hopewell, Va. and SILWET™ series, available from OSI Specialties, Inc., Danbury, Conn. The dimethicone copolyol has about 15 or fewer ethylene oxide and/or propylene oxide monomer units, in total, in the side chains.

Suitable silicone surfactants for use in compositions of the invention are disclosed in U.S. Pat. No. 4,122,029 to Gee which is hereby incorporated by reference. Another exemplary, but nonlimiting, oil-soluble, silicon- based surfactant is an alkyl dimethicone copolyol, such as cetyl dimethicone copolyol available commercially as ABIL EM from Goldschmidt Chemical Corporation, Hopewell, Va.

Optional Aerosol Propellant

The hair compositions of the invention are dispensed as a foam. They can be dispensed from nonaerosol foaming packages as described herein. When they are dispensed as aerosol foams then they are dispensed using a conventional aerosol propellant such as trichlorofluoromethane, dichlorodifluoromethane, difluoroethane, dimethylether, propane, n-butane or isobutane or mixtures thereof.

Aqueous Carrier

The carrier of the styling composition is predominantly water. Deionized water can be employed.

Optional Ingredients which may be used in Compositions of the Invention

In addition to the above-described ingredients, other common cosmetic components and additives can be incorporated in the compositions of the invention, as long as the basic properties of the composition, such as an ability to cleanse the hair, are not adversely affected. Such optional ingredients include, but are not limited to, humectants, inorganic salts, fragrances, hydrotropes, preservatives, water softening agents, acids, bases, buffers and the like. Optional components usually are present in weight percentages of less than about 2% each, and from about 5% to about 10% by weight of the composition in total.

Other optional materials that can be used in compositions of the invention include fatty esters, polymers, such as styrene polymers, and fatty alcohols.

Foaming Packages or Containers which may be used with Compositions of the Invention

In order to conveniently produce the aerated foams of this invention, a suitable non-pressurized foam container such as that described in U.S. Pat. No. 3,709,437 which is hereby incorporated by reference, can be employed. The composition can be placed into the reservoir of a plastic squeeze bottle which contains a foamer head or other foam producing means. Squeezing the container causes the solution to leave the reservoir and enter an air-mixing or foaming chamber via an internal dip tube. The foam produced in the foaming chamber is often passed through a homogenizing element interposed between the air-mixing chamber and the discharge orifice to homogenize and control the consistency of the discharged foam. Further compression of the foam discharges the foam from a discharge cap as a uniform non-pressurized aerated foam. Alternatively, the side walls of the container may be rigid and the dip tube may be fitted with a pump that is actuated by a push button. When composition is drawn by the pump through to the air mixing or foaming chamber, the desired foam is produced.

Other means for producing foams will be apparent to those skilled in the art. Means for producing aerated foams are described in U.S. Pat. Nos. 4,511,486 and 4,018,364 both of which are hereby incorporated by reference.

Without intending any limitation on the types of foaming devices which may be employed in this invention, the applicants have employed a type of foamer that is called an F2 Pumpfoamer. This kind of pump foaming device is manufactured by Airspray, of Pompano Beach, Fla. This device is actuated by a push button and supplies 0.75 ml of composition/stroke or push +/−0.05 ml. Of course, the consumer can control the amount of foam placed on his or her hair through the use of the pump foaming device, by the number of strokes of the push button.

The compositions of the invention can be altered to work more efficiently with the particular foaming package or container being employed. One skilled in the art would know how to make such changes within the scope of the present invention.

In addition, pressurized foam containers which contain a propellant as described above may be used to dispense compositions of the invention. Such pressurized containers are conventional in the art and are described, for example, in the following patents which are hereby incorporated by reference: U.S. Pat. No. 5,388,620 and U.S. Pat. No. 4,893,730.

How to use Compositions of the Invention

To shampoo the hair, compositions of the invention may be applied as a foam to hair that is dry. Water can be added and the foam can be worked into the hair, usually with the fingers. The foam is then rinsed from the hair. Or the foam may be added to hair that is already wet and worked into the hair usually with fingers, with or without additional water, and then rinsed from the hair. In fact, the compositions of the invention may be used in any manner that a conventional shampoo is used except that it is dispensed in the form of a foam.

As noted above, a foam is easier for a child or an infant to handle than is a traditional liquid shampoo. Moreover, a foam is more easily kept from the eyes than is a liquid shampoo. Finally, a foam is more fun for a child or infant to use that than is a liquid, because a foam is light and fluffy in appearance.

Compositions of the invention may be made by methods which are known in the are or which are analogous to methods which are known in the art.

Anionic surfactant blends of the invention may be made by methods which are known in the art or which are analogous to methods which are known in the art.

The following examples show compositions of the invention. These examples are illustrative of compositions of the invention; however, the invention is not limited by these examples.

The following examples were made.

### EXAMPLE 1

| Ingredient | Weight Percent |
|---|---|
| Deionized Water | 59.0784 |
| Polyquaternium 10, 93% Active | 0.0096 |
| Sodium Trideceth Sulfate, 30% Active | 13.104 |
| PEG 80 Sorbitan Laurate, 72% Active | 6.384 |
| Cocamidopropyl Hydroxysultaine, 44% Active | 3.8976 |
| Disodium Lauramphoacetate, 30% Active | 3.36 |
| PEG-150 Distearate, 100% Active | 1.008 |
| Sodium Laureth-13 Carboxylate, 67% Active | 0.672 |
| Glycerin, 95% Active | 1.2 |
| Liquid Citric Acid, 50% Active | 0.336 |
| Sodium Benzoate, 100% Active | 0.48 |
| Polysorbate 20, 97% Active | 4.8 |
| Dimethicone Copolyol, 100% Active | 0.96 |
| Fragrance, 100% Active | 0.48 |
| Methylchloroisothiazolinone | 0.048 |
| (and) Methylisothiazolinone, 1.5% Active | |
| DMDM Hydantoin, 55% Active | 0.096 |
| A-46 Propellant: | |
| Isobutane, 85% | 3.4 |
| Propane, 15% | 0.6 |

Method for Making this Formulation:

1. Add Deionized Water to the manufacturing tank, between 70-90 F.

2. Add Polyquaternium-10 slowly into vortex. Mix until clear and free of lumps.

3. Heatto 130 F.

4. Add surfactant blend and mix until homogeneous, maintaining a batch temperature of 130 F.

5. Add Glycerine.

6. Add Liquid Citric Acid.

7. Begin cooling batch to 95 F. Add the following ingredients: Fragrance, Methylchloroisothiazoline (and) Methylisothiazolinone, and DMDM Hydantoin.

8. Below 90 F., add concentrate to the can and vacuum crimp. Using either under the cup or through the valve method, fill can with propellant.

### EXAMPLE 2

| Ingredient | Weight Percent |
|---|---|
| Deionized Water | 52.93632 |
| Polyquaternium 10, 93% Active | 0.192 |
| Sodium Trideceth Sulfate, 30% Active | 17.9712 |
| PEG 80 Sorbitan Laurate, 72% Active | 8.7552 |
| Cocamidopropyl Hydroxysultaine, 44% Active | 5.34528 |
| Disodium Lauramphoacetate, 30% Active | 4.608 |
| PEG-150 Distearate, 100% Active | 1.3824 |
| Sodium Laureth-13 Carboxylate, 67% Active | 0.9216 |
| Glycerin, 95% Active | 1.2 |
| Liquid Citric Acid, 50% Active | 0.336 |
| Sodium Benzoate, 100% Active | 0.48 |
| Polysorbate 20, 97% Active | 0.96 |
| Dimethicone Copolyol, 100% Active | 0.96 |
| Fragrance, 100% Active | 0.48 |
| Methylchloroisothiazolinone | 0.048 |
| (and) Methylisothiazolinone, 1.5% Active | |
| DMDM Hydantoin, 55% Active | 0.096 |
| A-46 Propellant: | |
| Isobutane, 85% | 3.4 |
| Propane, 15% | 0.6 |

Method for Making this Formulation:

1. Add Deionized Water to the manufacturing tank, between 70-90 F.

2. Add Polyquaternium-10 slowly into vortex. Mix until clear and free of lumps.

3. Heatto 130 F.

4. Add surfactant blend and mix until homogeneous, maintaining a batch temperature of 130 F.

5. Add Glycerine.

6. Add Liquid Citric Acid.

7. Begin cooling batch to 95 F. Add the following ingredients: Fragrance, Methylchloroisothiazoline (and) Methylisothiazolinone, and DMDM Hydantoin.

8. Below 90 F., add concentrate to the can and vacuum crimp. Using either under the cup or through the valve method, fill can with propellant.

Properties and Benefits of Compositions of the Invention

The skin mildness properties of the foamable compositions of the present invention can be proven by the soap chamber test. The lack of irritation to the eyes of the foamable compositions of the present invention can be proven by the Flourecein Leakage Assay test.

What is claimed is:

1. A composition comprising:
(i) about 0.005 to about 5% of a cationic deposition polymer having a cationic charge density of about 0.1 to about 4.0 meq/gram;
(ii) about 10 to about 20% of an anionic surfactant which is selected from the group consisting of an alkyl ether sulfate with at least about two moles of ethoxylation, and a neutralized alkyl ether sulfate with at least about two moles of ethoxylation; and mixtures thereof;
(iii) about 0.5 to about 1.0% of an organic salt of a carboxylic acid;
(iv) about 6 to 15% sorbitan derivative;
(v) about 3 to about 6% of a zwitterionic, surface active compound;
(vi) about 0.25 to about 5% of an amphoteric surfactant;
(vii) about 0.75 to about 1.5% of an alkoxylated carboxylic acid
(viii) about 0.1 to about 5% of a silicone copolyol
(ix) optionally an aerosol propellant; and
(x) water.

2. A foamable, shampoo composition according to claim 1 which comprises:
(i) about 0.1 to about 1% of a cationic deposition polymer having a cationic charge density of about 0.1 to about 4.0 meq/gram;
(ii) about 12 to about 18% of an anionic surfactant which is selected from the group consisting of an alkyl ether sulfate with at least about two moles of ethoxylation, and a neutralized alkyl ether sulfate with at least about two moles of ethoxylation; and mixtures thereof;
(iii) about 0.6 to about 0.9% of an organic salt of a carboxylic acid;
(iv) about 6 to about 15% of sorbitan derivative;
(v) about 4 to about 5% of a zwitterionic, surface active compound;
(vi) about 3 to about 5% of an amphoteric surfactant;
(vii) about 0.75 to about 1.5% of an alkoxylated carboxylic acid
(viii) about 0.25 to about 1 % of a silicone copolyol
(ix) optionally an aerosol propellant; and
(x) water.

3. A composition according to claim 1 which is dispensed as a foam from an aerosol can.

4. A composition according to claim 1 which is dispensed as a foam from an nonaerosol can.

5. A composition according to claim 1 which is dispensed as a foam which is non-irritating to the skin and eyes.

6. A method for cleansing hair which comprises contacting said hair with a composition according to claim 1.
